(19) United States
(12) Patent Application Publication
(10) Pub. No.: US 2026/0183210 A1
(43) Pub. Date: Jul. 2, 2026

(54) COSMETIC COMPOSITION COMPRISING AT LEAST ONE VEGETABLE OIL, TRIHYDROXYSTEARIN AND A PIGMENTARY COLORING MATERIAL

(71) Applicant: L'OREAL, Paris (FR)

(72) Inventor: Alexandra SCHVENT, Chevilly la Rue (FR)

(73) Assignee: L'OREAL, Paris (FR)

(21) Appl. No.: 19/131,626

(22) PCT Filed: Nov. 24, 2023
(86) PCT No.: PCT/EP2023/082952

§ 371 Date: May 21, 2025

(30) Foreign Application Priority Data
Nov. 25, 2022 (FR) FR2212321

(51) Int. Cl. A61K 8/37; A61K 8/92; A61Q 1/04; A61Q 1/08

(52) U.S. Cl. CPC A61K 8/375; A61K 8/922; A61Q 1/04; A61Q 1/08; A61K 2800/43; A61K 2800/87

(57) ABSTRACT

This invention relates to a cosmetic composition, in particular make-up and/or treatment intended to be applied on the skin, lips and skin appendages of humans such as hair, eyelashes, eyebrows or nails, comprising glyceryl trihydroxystearate, at least 50% by weight with respect to the total weight of the composition of at least one vegetable oil and at least one pigmentary coloring material. Another object of the invention is a method for making up and/or treating the skin and/or lips and/or cheeks, in particular lips and/or cheeks, wherein the composition according to the invention is applied.

This invention relates to a cosmetic composition, in particular make-up and/or treatment intended to be applied on the skin, lips and skin appendages of humans such as hair, eyelashes, eyebrows or nails, comprising glyceryl trihydroxystearate, at least 50% by weight with respect to the total weight of the composition of at least one vegetable oil and at least one pigmentary coloring material. Another object of the invention is a method for making up and/or treating the skin and/or lips and/or cheeks, in particular lips and/or cheeks, wherein the composition according to the invention is applied.

Cosmetic compositions intended for treatment and/or make-up, in particular for the lips, have been known for very long time and come in more and more varied forms, ranging from formulae of the viscous fluid type such as glosses, to solid compositions in stick form, supported or not, compositions in pencil form, or compositions stored in pots.

Historically, compositions for the lips are in general anhydrous but, in the past few years, make-up compositions for the lips in emulsion form have been seen to appear.

For the purpose of further diversifying the range of products, transparent anhydrous liquid formulae more fluid than conventional glosses have appeared on the market, comprising high oil contents.

However, because of the very nature of this type of composition, introducing sufficient quantities of pigments, to obtain compositions that remain stable or easily rehomogenizable before application and are easy to apply in comfortable and non-sticky deposits, is complex.

Likewise, in the case of application to the cheeks, it is desirable to obtain make-up and/or treatment compositions (for example of the blusher type) that are non-sticky and non-glossy and have satisfactory rendition.

In the application WO 2022/003026, liquid make-up compositions applicable to the skin and/or lips are described, comprising, apart from particulate colored materials, trihydroxystearin, a polar wax, and high proportions (i) of polyester oil derived from dimerized acid, and (ii) of polar oils selected from triglycerides and vegetable oils. Such compositions, which are fairly viscous, make it possible to obtain a deposit the stickiness of which is reasonable, very glossy with good retention of this glossiness over time. However, it may be desirable to have compositions the deposition of which is fine, less “present” on the skin and lips, less sticky also, and the rendition of which is non-glossy to satin.

There is therefore a need for make-up compositions that can be used both on the skin, for example the cheeks, and the lips, which do not stick and which are easy to apply. Difficulties are however encountered with this type of composition, in particular because the proportion of vegetable oils, which are oils of low viscosity, is very high, and because the composition contains particles, in particular coloring materials such as pigments.

This is because, under certain conditions, rehomogenizing the compositions before application thereof is not simple. It can be envisaged using stirring means in the receptacle (such as for example beads). However, these devices are not always usable, and effectively, in particular when the compositions are very viscous/gelled. The use of beads is also not always facilitated or possible, depending on the dimensions of the receptacle in which the composition is present, depending on the presence or not of an immersed applicator. Finally, if the compositions are non-viscous/gelled, the pigments the sedimentation of which is less interfered with by the composition, can give a more cohesive deposit that is more difficult to rehomogenize.

It is desirable to obtain compositions that are easily rehomogenizable before application, without loss of their usage properties. In particular, these compositions must be able to be applied easily in a precise, non-sticky and homogenous deposit, which does not flow outside the made-up area.

Finally, the cosmetic-product formulation comprising high proportions of natural raw materials or raw materials of natural origin represents one of the new challenges for satisfying the expectations of consumers. It is therefore desirable for the compositions proposed to make it possible to keep a high proportion of natural raw materials or raw materials of natural origin.

An object of the present invention is therefore to provide a solution to the above problems.

The inventors have developed a fluid colored composition, the oily phase of which is in particular transparent or translucent, which comprises a high proportion of vegetable oil or oils and glyceryl trihydroxystearate, which is stable or easily rehomogenizable, easy to apply and non-sticky. This composition can in particular be applied to the skin and/or lips and/or cheeks. It leaves a non-sticky, non-glossy and satin deposit, and has satisfactory rendition. It is easily rehomogenizable before application.

Its object is thus a cosmetic composition, in particular for make-up and/or treatment of the skin and/or lips, in particular the lips, comprising:
glyceryl trihydroxystearate;
at least 50% by weight, relative to the total weight of composition, at least one vegetable oil; and
at least one pigmentary coloring material.

Another object of the invention is a method for making up and/or treating the skin and/or lips and/or cheeks, in particular lips and/or cheeks, wherein the composition according to the invention is applied to the skin and/or lips and/or cheeks.

The cosmetic composition according to the invention is non-sticky, and can be applied both to the skin, in particular the cheeks, and the lips. It is easily rehomogenizable before application if required, without loss of its usage properties.

Finally, it comprises a high proportion of natural raw materials or raw materials of natural origin.

The composition according to the invention is more particularly in a fluid form.

“Fluid” means a particular composition where it is possible to measure a viscosity, in particular in accordance with the following protocol.

### Protocol for Measuring Viscosity:

The viscosity is generally measured at 25° C., using a Rheomat RM 200 viscometer equipped with a suitable spindle, in particular n°2, the measurement being made after 10 minutes of rotation of the spindle in the composition, at a shear rate of 200 revolutions/min (rpm).

In particular, the viscosity of the composition according to the invention at 25° C. is less than or equal to 1 Pa·s, preferably less than or equal to 0.7 Pa·s, and advantageously between 0.4 and 0.6 Pa·s.

Preferably, the composition according to the invention is substantially free from liquid polyester obtained from an unsaturated fatty acid dimer, the fatty acid comprising from 16 to 22 carbon atoms, with a diol. “Substantially free from polyester” means that the composition according to the invention comprises less than 1% by weight polyester, preferably less than 0.5% by weight, preferably less than 0.1% by weight, in relation to the total weight of composition. Preferably, the composition according to the invention is completely free from liquid polyester obtained from an unsaturated fatty acid dimer, the fatty acid comprising from 16 to 22 carbon atoms, with a diol.

Liquid polyester means a polyester that begins to flow under its own weight in less than one minute at ambient temperature (25° C.). “Unsaturated fatty acid” designates fatty acids comprising 1 to 6 unsaturations (double carbon-carbon bond); mention can be made in particular of palmitoleic acid, oleic acid, linoleic acid, elaidic acid, gadoleic acid, eicosapentaenoic acid, erucic acid, brassidic acid, arachidonic acid and mixtures thereof.

The unsaturated fatty acid dimer is more particularly obtained by polymerization reaction, in particular intermolecular dimerization of at least one unsaturated monocarboxylic acid. It derives in particular from the dimerization of an unsaturated fatty acid, in particular C16-C22, and more particularly C18.

Advantageously, the composition according to the invention is substantially free from liquid polyester obtained from a diacid dimer obtained by dimerization of linoleic acid, optionally followed by hydrogenation of the carbon-carbon bonds, total or partial, advantageously total.

Advantageously, the composition according to the invention is substantially free from liquid polyester obtained by reacting the aforementioned acid dimer with at least one diol, more particularly C3-C4, preferably linear, preferably saturated. Particularly preferably, the diol is 1,2-propanediol, 1,3-propanediol, 1,2-butanediol, 1,3-butanediol or 1,4-butanediol, and preferably 1,3-propanediol or 1,4-butanediol.

Advantageously, the composition according to the invention is substantially free from liquid polyester obtained by condensing dilinoleic acid and 1,4-butanediol, or dilinoleic acid and 1,3-butanediol. Such a liquid polyester is in particular dilinoleic acid/butanediol copolymer (INCI name) marketed in particular by Biosynthis under the name Viscoplast® 14436H, or dilinoleic acid/propanediol copolymer (INCI name) marketed in particular by Biosynthis under the name Viscoplast® Green 3000.

Advantageously, the composition according to the invention is substantially free from liquid polyester selected from those resulting from the dilinoleic dimer acid reaction, with at least propanediol or butanediol.

### Glyceryl Trihydroxystearate

As indicated previously, the composition according to the invention comprises glyceryl trihydroxystearate.

This compound is a total ester of hydroxystearic acid (also called 12-hydroxystearic acid) and glycerol (and therefore a triglyceride). The INCI name of this compound is trihydroxystearin. It is for example marketed under the name Thixcin® R or Thixcin® E, by the company Elementis Specialties.

This compound is used in the present invention as a gelling agent rather than as a wax. In other words, it is advantageously used, in the method for preparing the composition, at a temperature that remains below that of its melting point (around 55-60° C.).

Preferably, the composition comprises between 0.5 and 4% by weight in relation to the total weight of the composition, more particularly between 1 and 4% by weight, and even more advantageously between 1.5 and 3% by weight.

### Vegetable Oils

The composition according to the invention furthermore comprises at least one vegetable oil.

Vegetable oils are non-volatile polar hydrocarbon oils.

“Non-volatile oil” designates compounds liquid at 25° C. and atmospheric pressure (1.013.10⁵ Pa), the vapor pressure of which at 25° C. is not zero and is less than 2.66 Pa, more particularly less than 0.13 Pa. By way of example, the vapor pressure can be measured in accordance with the static method or by the isothermal thermogravimetry effusion method, according to the vapor pressure (OCDE 104).

The term “polar hydrocarbon oil” refers to an oil essentially formed, or consisting, of carbon and hydrogen atoms, and also comprising at least one oxygen, and optionally nitrogen, atom. These oils are therefore distinct from silicone oils.

Among the vegetable oil or oils suitable for implementing the present invention, mention can be made of jojoba oil, castor oil, olive oil, coconut oil, DHA algal oil, ximenia oil, pracaxi oil, coriander seed oil, macadamia oil, passionflower oil, argan oil, sesame oil, sunflower oil, grapeseed oil, avocado oil, Rosa canina oil, apricot kernel oil, flax oil, and polymerized flax oil, sweet almond oil, cotton seed oil, soybean oil, rapeseed oil, canola oil, groundnut oil, kaya oil, coconut oil, Marula oil, Camelina oil, wheatgerm oil, corn oil, corn germ oil, rice bran oil, alfalfa oil, poppy seed oil, ambercup squash oil, marrow oil, hazelnut oil, grapeseed oil, blackcurrant oil, evening primrose oil, millet oil, barley oil, quinoa oil, rye oil, safflower oil, candleberry oil, limnanthes oil, black cumin oil, buriti oil, sandal nut oil, Babassu oil, the liquid shea butter fraction, the liquid cocoa butter fraction, and mixtures thereof.

Preferably, the composition according to the invention comprises a mixture of vegetable oils. Preferably, the composition according to the invention comprises castor oil, rapeseed oil and/or canola oil.

The composition according to the invention comprises at least 50% by weight, with respect to the total weight of the composition, at least one vegetable oil, preferably at least 60% by weight, with respect to the total weight of the composition.

Preferably, the composition comprises between 50 and 95% by weight of at least one vegetable oil, more particularly between 55 and 90% by weight, and even more advantageously between 60 and 85% by weight with respect to the total weight of the composition.

### Additional Liquid Compounds

The composition according to the invention can optionally comprise at least one additional oil, non-volatile or volatile, hydrocarbon or silicone, different from the vegetable oils previously described.

The term “silicone oil” refers to an oil (compound liquid at 25° C. and atmospheric pressure (1.013.10⁵ Pa)) containing at least one silicon atom and particularly containing Si—O groups.

The term “volatile oil” refers to an oil that has a non-zero vapor pressure, at ambient temperature and atmospheric pressure, ranging in particular from 2.66 Pa to 40,000 Pa, in particular ranging from 2.66 Pa to 13,000 Pa, and more particularly ranging from 2.66 Pa to 1,300 Pa.

### Additional Non-Volatile Polar Hydrocarbon Oils

Preferably, the composition according to the invention comprises at least one additional non-volatile polar hydrocarbon oil different from the vegetable oils previously described.

“Polar oil” within the meaning of the invention designates compounds liquid at 25° C. and atmospheric pressure (1.013.10⁵ Pa), comprising, in addition to the carbon and hydrogen atoms, at least one oxygen or nitrogen atom, and preferably at least one oxygen atom.

By way of non-volatile polar hydrocarbon oils different from the plant oils described previously, mention can be made of monoesters or polyesters comprising at least 17 carbon atoms. In all cases, these polyesters comprising at least 17 carbon atoms are different from triglycerides.

As examples, mention can be made of mono-, di- or tri- or tetra-esters. The non-volatile polar hydrocarbon oil different from the vegetable oils described previously can be selected for example from:
monoesters comprising between 17 and 40 carbon atoms in total, in particular monoesters, having formula RICOOR2 wherein R₁ is the remainder of a linear or branched or aromatic fatty acid including from 4 to 40 carbon atoms, saturated or not, and R₂ is a hydrocarbon chain in particular branched containing from 3 to 40 carbon atoms with the condition that R₁+R₂ is greater than or equal to 17, such as for example Purcellin oil (cetostearyl octanoate), isononyl isononanoate, C12 to C15 alcohol benzoate, 2-ethyl hexyl palmitate, octyldodecyl neopentanoate, octyl-2-dodecyl stearate, octyl-2-dodecyl erucate, isostearyl isostearate, octyl-2 dodecyl benzoate, octanoates, decanoates or ricinoleates of alcohols or of polyalcohols, isopropyl myristate, isopropyl palmitate, butyl stearate, hexyl laurate, 2-ethyl-hexyl palmitate, 2-hexyl-decyl laurate, 2-octyl-decyl palmitate, 2-octyldodecyl myristate. Preferably, these are esters having formula RICOOR2 wherein R₁ is the remainder of a linear or branched fatty acid including from 4 to 40 carbon atoms and R₂ is a hydrocarbon chain in particular branched containing from 3 to 40 carbon atoms, with R₁ and R₂ being such that R₁+R₂ is ≥17. Even more particularly, the ester comprises between 17 and 40 carbon atoms in total. As preferred monoesters, mention can be made of isononyl isononanoate, oleyl erucate and/or octyl-2-docecyl neopentanoate;
diesters, in particular comprising between 18 and 60 carbon atoms in total, in particular between 18 and 50 carbon atoms in total. In particular diesters of carboxylic diacid and of monoalcohols can be used, such as preferably diisostearyl malate, or the diesters of glycol and of monocarboxylic acids, such as neopentylglycol diheptanoate, propylene glycol dioctanoate, diethylene glycol diisononanoate, or polyglyceryl-2 diisostearate (in particular such as the compound sold under the commercial reference Dermol DGDIS by Alzo);
triesters, in particular comprising between 25 and 80 carbon atoms in total, preferably from 25 to 70 carbon atoms, alone or in mixtures, in particular such as triesters of carboxylic triacid, such as triisostearyl citrate, or tridecyl trimellitate; C₈-C₆₀, preferably C₈-C₄₀, acid triglycerides, in particular saturated, such as caprylic/capric triglycerides, C18-36 acid triglycerides, glyceryl triisostearate (triisostearin), or 2-decyl-1-tetradecanol triglyceride. It should be noted that caprylic/capric acid triglycerides are for example marketed under the range DUB MCT by the company Stearinerie Dubois, C18-36 acid triglycerides are in particular marketed under the reference DUB TGI 24 by Stéarinerie Dubois. The following are also suitable: polyglycerol and carboxylic monoacid triesters, in particular branched, such as polyglycerol-2 triisostearate;
tetraesters, in particular having a total number of carbon of at least 35, such as tetraesters of penthaerythritol or of polyglycerol and of a monocarboxylic acid, for example such as pentaerythrityl tetrapelargonate, pentaerythrityl tetraisostearate, pentaerythrityl tetraisononanoate, polyglyceryl-2 tetraisostearate, pentaerythrityl tetradecyl-2 tetradecanoate or mixtures thereof.

Preferably, the composition according to the invention comprises at least one additional non-volatile polar hydrocarbon oil different from the vegetable oils described previously, selected from triesters comprising between 25 and 80 carbon atoms in total, preferably between 25 and 70 carbon atoms in total, in particular such as caprylic/capric triglycerides, triesters of polyglycerol and of branched monocarboxylic acids such as polyglycerol-2 triisostearate, and mixtures thereof. Preferably, the concentration of additional non-volatile polar hydrocarbon oil or oils different from the vegetable oils described previously is between 1 and 45% by weight, preferably between 5 and 40% by weight, preferably between 10 and 35% by weight with respect to the total weight of the composition.

According to a particular embodiment of the invention, the concentration of vegetable oil or oils and of additional non-volatile polar hydrocarbon oil or oils represents at least 50% by weight, more particularly at least 60% by weight, even more preferentially at least 70% by weight, with respect to the total weight of the composition; preferably between 50 and 98% by weight, more particularly between 60 and 97% by weight, and even more advantageously between 75 and 95% by weight with respect to the total weight of the composition.

According to a particularly advantageous variant of the invention, the composition comprises a mixture of vegetable oils, preferably a mixture of castor oil, rapeseed oil, canola oil and caprylic/capric triglycerides.

### Additional Non-Polar Non-Volatile Hydrocarbon Oils

The term “non-polar oil”, in terms of this invention, refers to an oil selected from hydrocarbons, i.e. from liquid compounds comprising only carbon and hydrogen atoms.

More particularly, the non-polar non-volatile hydrocarbon oil can be selected from linear or branched hydrocarbons, of mineral, vegetable or synthetic origin, such as for example polybutenes, polyisobutylenes, polydecenes, hydrogenated or not, mixtures of linear saturated hydrocarbons, more particularly C₁₅-C₂₈, squalane, in particular of plant origin, or paraffin oils.

### Additional Non-Volatile Silicone Oils

The non-volatile silicone oils can be non-phenylated, or phenylated comprising or not at least one dimethicone fragment.

The term “phenylated” specifies that said oil includes in its structure at least one phenyl radical.

The term “dimethicone fragment” designates a divalent siloxane group of which the silicon atom carries two methyl radicals, with this group not being located at one and or both ends of the molecule. It can be represented by the following formula: —(Si(CH₃)₂—O)—.

Preferably, the silicones do not contain a C₂-C₃ alkylene oxide group or a glycerol group.

Among non-volatile non-phenylated silicones, mention can be made of polydimethylsiloxanes (INCI name: dimethicone), alkyldimethicones comprising at least one C₂-C₂₄ alkyl group, and mixtures thereof.

Among non-volatile phenylated silicones containing at least one dimethicone fragment, mention can be made of compounds with the following INCI names: Trimethylsiloxyphenyl Dimethicone, Diphenyl Dimethicone, Tetramethyl Tetraphenyl Trisiloxane and mixtures thereof.

With regard to non-volatile siliconated phenylated silicones, with no dimethicone fragment, mention can be made of the compounds with the following INCI names: Phenyltrimethicone and Trimethyl Pentaphenyl Trisiloxane, alone or in mixtures.

### Additional Volatile Silicone or Hydrocarbon Oils

By way of examples of volatile silicone oils, mention can be made in particular of dimethicones with a viscosity of less than 5 cSt (5×10⁻³ mm²/s, measured in particular according to ASTM D-445), octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

Among volatile hydrocarbon oils, preferably non-polar, mention can be made of volatile hydrocarbon oils having from 8 to 16 carbon atoms and mixtures thereof, and in particular:
C₈-C₁₆ or C₈-C₁₆ branched alkanes such as iso-alkanes (also called isoparaffins), isododecane, isodecane, isohexadecane,
linear alkanes, preferably for example such as the mixture of n-decane (C10) and n-dodecane (C12) sold by Biosynthis under the reference Vegelight silk, n-dodecane (C12) and n-tetradecane (C14) sold by Sasol respectively under the references Parafol 12-97 and Parafol 14-97, the undecane-tridecane mixture (Cetiol UT), the mixtures of n-undecane (C11) and n-tridecane (C13) obtained in examples 1 and 2 of the application WO2008/155059 of Cognis, and mixtures thereof.

Preferably, if the composition comprises a silicone oil or oils, volatile or non-volatile, and/or one or more volatile or non-volatile non-polar hydrocarbon oil or oils, the proportion thereof does not exceed 5% by weight, preferably does not exceed 3% by weight, with respect to the weight of the composition.

In accordance with a particular embodiment of the invention, the composition does not comprise any additional volatile or non-volatile silicone oil or oils.

### Additional Solid Compounds

The composition according to the invention can optionally comprise at least one solid compound (solid at ambient temperature and atmospheric pressure), selected more particularly from hydrocarbon waxes, from pasty hydrocarbon compounds and mixtures thereof.

### Waxes

“Wax”, within the meaning of the invention, designates a lipophilic compound solid at ambient temperature (25° C.), having a melting temperature greater than or equal to 40° C. and less than or equal to 120° C., more particularly less than or equal to 90° C.

Within the meaning of the invention, the melting temperature (or melting point) is the temperature of the most endothermic peak observed in thermal analysis (DSC), as described in ISO 11357-3:1999.

The melting point of a solid fat can be measured using a differential scanning calorimeter (DSC), for example the calorimeter sold under the trade name “DSC 2000” by TA Instruments with the “TA Universal Analysis” software.

The measurement protocol is as follows:

A 5 mg sample of wax is placed in a crucible and subjected to a first temperature rise from −20° C. to 120° C., at a heating rate of 10° C./minute, and is then cooled from 120° C. to −20° C. at a cooling rate of 10° C./minute and finally subjected to a second temperature rise from −20° C. to 120° C. at a heating rate of 5° C./minute. During the second temperature rise, the melting point of the solid fat is measured, corresponding to the temperature of the most endothermic peak of the melting curve observed.

More particularly, the wax can be selected from polar waxes, different from glyceryl trihydroxystearate, in particular alcohol waxes; esters such as waxes of animal or plant origin, such as beeswax, lanolin, sunflower, candelilla, carnauba, rice bran, montan, hydrogenated jojoba oil; waxes resulting from the hydrogenation of esters obtained from C6-C22 fatty alcohols of plant origin and vegetable oil; waxes having the formula R₁COOR₂ wherein R₁ and R₂ are aliphatic linear, branched or cyclic chains wherein the number of atoms varies from 10 to 50, that can contain a heteroatom, in particular oxygen, and the melting temperature of which varies from 40 to 120° C.; partial or total, preferably total, esters of a C₁₆-C₃₀ carboxylic acid, saturated, with glycerol, such as glyceryl tristearate or tribehenate, and mixtures thereof.

Mention can also be made of non-polar hydrocarbon waxes (in other words consisting solely of carbon and hydrogen atoms), resulting from the transformation of petroleum, such as for example polyethylene waxes, polymethylene waxes (synthetic wax, Fischer-Tropsch waxes), alone or in mixtures.

If the composition comprises at least one wax, then the proportion of this type of compound does not exceed 1% by weight in relation to the total weight of said composition.

Preferably, the composition according to the invention is devoid of any wax compound or siliconated pasty compound.

### Pasty Compounds

“Pasty compound”, within the meaning of the present invention, means a lipophilic compound having at a temperature of 25° C. a liquid fraction and a solid fraction. Thus a pasty compound can have a commencing melting point of less than 25° C.

The melting point of the pasty fat is determined according to the same principle as that detailed previously for waxes. In the case of a pasty compound, the measurement protocol is however as follows:

A 5 mg sample of pasty fat placed in a crucible is subjected to a first temperature rise from −20° C. to 100° C., at a heating rate of 10° C./minute, and is then cooled from 100° C. to −20° C. at a cooling rate of 10° C./minute and finally subjected to a second temperature rise from −20° C. to 100° C. at a heating rate of 5° C./minute. The melting point of the pasty fat is the value of the temperature equivalent to the top point of the peak of the curve representing the variation in the difference in power absorbed as a function of temperature. It should be noted that the liquid fraction by weight of the pasty fat at ambient temperature is equal to the ratio of the enthalpy of fusion consumed at ambient temperature to the enthalpy of fusion of the pasty fat. The enthalpy of fusion of the pasty fat is the enthalpy consumed by the latter to change from the solid state to the liquid state. The pasty fat is said to be in the solid state when the entire mass thereof is in solid crystalline form. The pasty fat is said to be in the liquid state when the entire mass thereof is in liquid form. The enthalpy of fusion of the pasty fat is the quantity of energy required to change the pasty fat from the solid state to the liquid state. It is expressed in J/g. The enthalpy of fusion of the pasty fat is equal to the area under the curve of the thermogram obtained.

Preferably, this or these pasty hydrocarbon compounds are selected from vaseline; plant butters, such as in particular mango, shea, cupuacu, murumuru, cocoa, babassu and jojoba butters; completely or partially hydrogenated vegetable oils, such as for example hydrogenated soya oil, hydrogenated copra oil, hydrogenated rapeseed oil, mixtures of hydrogenated vegetable oils such as products with the INCI name hydrogenated vegetable oil, partially hydrogenated olive oil; esters of hydrogenated castor oil and of C₁₆-C₂₂ fatty acids, bis-diglyceryl polyacyladipate-2; products with the INCI name Hydrogenated Coco-Glycerides; esters of dilinoleic alcohol dimer and/or of dilinoleic acid such as for example products with the following INCI names: Bis-Behenyl/Isostearyl/Phytosteryl Dimer Dilinoleyl Dimer Dilinoleate, Phytosteryl/Isostearyl/Cetyl/Stearyl/Behenyl Dimer Dilinoleate), Hydrogenated Castor Oil Dimer Dilinoleate, and mixtures thereof.

If the composition according to the invention comprises any of them, then the proportion thereof does not exceed 5% by weight, preferably does not exceed 3% by weight, advantageously does not exceed 1% by weight, with respect to the total weight of the composition.

Preferably, the composition according to the invention is substantially free from wax. Preferably, the composition according to the invention is substantially free from pasty compound. Preferably, the composition according to the invention is substantially free from wax and pasty compound. “Substantially free” means that the composition contains less than 1% by weight, preferably less than 0.5% by weight, wax and/or pasty compound.

Preferably, the composition according to the invention is totally free from wax. Preferably, the composition according to the invention is totally free from pasty compound. Preferably, the composition according to the invention is totally free from wax and pasty compound.

### Pigmentary Coloring Materials

The composition according to the invention comprises a pigmentary coloring material. This coloring material is thus selected from powdery coloring materials such as mineral pigments, nacres and organic pigments.

The term “pigments” should be understood to mean white or colored, mineral or organic particles, which are insoluble in an aqueous solution and are intended for coloring the composition and/or the resulting deposit.

The coloring materials may be present, in the composition, at a content ranging from 0.1% to 10% by weight, with respect to the weight of the composition, preferably from 0.5% to 5% by weight, preferably from 0.8% to 3% by weight.

### Mineral Pigments

According to one particular embodiment, the pigments used according to the invention are selected from mineral pigments.

“Mineral pigment” means any pigment that complies with the definition in Ullmann's Encyclopedia in the Inorganic Pigment chapter. Mention can be made, among the mineral pigments useful in the present invention, of zirconium or cerium oxides, along with zinc, iron (black, yellow or red) or chromium oxides, manganese violet, ultramarine blue, chromium hydrate and iron blue, titanium dioxide, metallic powders such as aluminum powder and copper powder. The following mineral pigments can also be used: Ta2O5, Ti3O5, Ti2O3, TiO, ZrO2 in a mixture with TiO2, ZrO2, Nb2O5, CeO2, ZnS.

The size of the pigment useful in the context of the present invention is in general greater than 100 nm and may range up to 10 μm, preferably from 200 nm to 5 μm, and more preferably from 300 nm to 1 μm. According to a particular embodiment of the invention, the pigments have a size characterized by a D50 greater than 100 nm and may range up to 10 μm, preferably from 200 nm to 5 μm, and more preferably from 300 nm to 1 μm. The sizes are measured by measured by static diffusion of light by means of a commercial MasterSizer 3000® granulometer from Malvern, making it possible to apprehend the granulometric distribution of all the particles over a wide range that can range from 0.01 μm to 1000 μm. The data are processed on the basis of the classic Mie scattering theory. This theory is the most adapted for size distributions ranging from submicron to multi-micron, and makes it possible to determine an “effective” diameter of particles. This theory is in particular described in the work by Van de Hulst, H. C., “Light Scattering by Small Particles”, Chapters 9 and 10, Wiley, New York, 1957. D50 represents a maximum size that 50% by volume of the particles have.

In the context of the present invention, the mineral pigments are more particularly iron oxide and/or titanium dioxide. By way of example, mention can be made more particularly of titanium dioxide and iron oxide, coated with aluminum stearoyl glutamate for example marketed under the reference NAIR by Miyoshi Kasei.

As mineral pigments that can be used in the invention, nacres can also be cited.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic coloring material, and pearlescent pigments based on bismuth oxychloride. This may also involve mica particles at the surface whereof are superposed at least two successive layers of metal oxides and/or of organic coloring materials. By way of example of nacres, mention may also be made of natural mica coated with titanium oxide, with iron oxide, with natural pigment or with bismuth oxychloride. The nacres may more particularly possess a yellow, pink, red, bronze, orange, brown and/or copper color or glint.

Among the pigments that can be used according to the invention, mention can also be made of those with an optical effect different from a simple conventional hue effect, i.e. a unified and stabilized effect of the kind produced by conventional coloring materials, such as, for example, monochromatic pigments. For the purpose of the invention, “stabilized” means absence of an effect of variability of color with the angle of observation or in response to a temperature change. For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

According to one particular embodiment, the composition according to the invention comprises at least one non-coated pigment.

According to another particular embodiment, the composition according to the invention comprises at least one pigment coated with at least one lipophilic or hydrophobic compound. This type of pigment is particularly advantageous. Since they are treated by a hydrophobic compound, they manifest a preponderant affinity for an oily phase, which can then convey them. The coating may also comprise at least one additional non-lipophilic compound.

According to the invention, the “coating” of a pigment according to the invention generally denotes the full or partial surface treatment of the pigment by a surface agent, absorbed, adsorbed or grafted onto said pigment.

The surface-treated pigments may be prepared using chemical, electronic, mechano-chemical or mechanical surface treatment techniques well known to those skilled in the art. Commercial products may also be used.

The surface agent may be absorbed, adsorbed or grafted onto the pigments by means of solvent evaporation, chemical reaction or creation of a covalent bond. According to a variant, the surface treatment consists of a coating of the pigments. The coating may represent 0.1% to 20% by weight and particularly 0.5% to 5% by weight of the total weight of the coated pigment. The coating may be carried out for example by adsorbing a liquid surface agent on the surface of the solid particles merely by mixing while stirring the particles and said surface agent, optionally heated, before incorporating the particles in the other ingredients of the composition. The coating may be carried out for example by means of a chemical reaction of a surface agent with the surface of the solid pigment particles and the creation of a covalent bond between the surface agent and the particles. This method is in particular described in the patent U.S. Pat. No. 4,578,266. The chemical surface treatment may consist in diluting the surface agent in a volatile solvent, dispersing the pigments in this mixture, and then slowly evaporating the volatile solvent, so that the surface agent is deposited on the surface of the pigments.

According to a particular embodiment of the invention, the pigments may be coated according to the invention with at least one compound selected from silicone surfactants; fluorinated surfactants; fluorosilicone surfactants; metal soaps, n-acyl amino acids or salts thereof; lecithin and its derivatives; isopropyl triisostearyl titanate; isostearyl sebacate; plant or animal natural waxes; polar synthetic waxes; fatty esters; phospholipids; and mixtures thereof.

According to one particular embodiment of the invention, the pigments can be coated with a hydrophilic compound.

### Organic Pigments

According to another embodiment of the invention, the pigmentary coloring material is an organic, synthetic or natural pigment or one of natural origin.

“Organic pigment” means any pigment that complies with the definition in Ullmann's Encyclopedia in the Organic Pigment chapter. The organic pigments can in particular be selected from the compounds nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, quinophthalone.

The organic pigment or pigments can be selected for example from carmine, carbon black, aniline black, melanin, azo yellow, quinacridone, phthalocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in patent FR 2 679 771.

The pigments can also be in the form of composite pigments as described in patent EP 1 184 426. These composite pigments may be composed in particular of particles including an inorganic core covered at least partially with an organic pigment and at least one binder for fixing the organic pigments to the core.

The pigment may also be a lacquer.

The term lacquer means insoluble coloring materials adsorbed on insoluble particles, the assembly thus obtained remaining insoluble during use.

The inorganic substrates onto which the coloring materials are adsorbed are for example alumina, silica, calcium and sodium borosilicate or calcium aluminum borosilicate, and aluminum.

Among the organic coloring materials, mention can be made of cochineal carmine. The products known under the following names can also be cited: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 1 O (CI 77 002), D&C Green 3 (CI 42 053), D&C Blue 1 (CI 42 090). By way of examples of lacquers, the product known by the name D&C Red 7 (CI 15 850:1) can be cited.

### Additional Coloring Materials

The composition according to the invention can optionally comprise at least one coloring material selected from preferably liposoluble coloring materials.

The term “coloring materials” refers to generally organic compounds, natural or synthetic, soluble in an oily phase or in solvents miscible in the oily phase, or in an aqueous or hydroalcoholic phase, and able to color.

### Liposoluble Coloring Material

The term “liposoluble coloring material” refers to in, terms of the invention, any generally organic compound, natural or synthetic, soluble in an oily phase or solvents miscible in the oily phase and able to color.

By way of liposoluble coloring materials suitable for the invention, the following liposoluble coloring materials can in particular be cited, such as for example DC Red 17, DC Red 21, DC Red 27, DC Green 6, DC Yellow 11, DC Violet 2, DC Orange 5, Sudan red and Sudan brown.

By way of illustration of natural liposoluble coloring materials, the following can in particular be cited: carotenes such as β-carotene, α-carotene, lycopene; quinoleic yellow; xanthophylls such as astaxanthin, antheraxanthin, citranaxanthin, cryptoxanthin, canthaxanthin, diatomoxanthin, flavoxanthin, fucoxanthin, lutein, rhodoxanthin, rubixanthin, siphonaxanthin, violaxanthin, zeaxanthin; rocou; curcumin; quinizarine (Ceres Green BB, D&C Green No. 6, CI 61565, 1,4-di-p-toluidinoanthraquinone, Green No. 202, Quinzarine Green SS) and chlorophylls.

### Alcohol/Polyol

The compositions according to the invention preferably comprise at least one saturated alcohol, linear or branched, comprising 2 to 8 carbon atoms, in particular 2 to 6 carbon atoms and in particular 2 to 4 carbon atoms, and comprising one or two hydroxyl (OH) functions.

The aliphatic monohydroxylated alcohol or alcohols can in particular be selected from ethanol, propanol, butanol, isopropanol, isobutanol or mixtures thereof.

The dihydroxylated alcohols (diols) are more particularly selected from glycols such as ethylene glycol, propylene glycol, pentylene glycol, caprylyl glycol, 1,3-butylene glycol and dipropylene glycol.

Preferably, the composition comprises at least one diol.

If the composition comprises such, the alcohol content varies more particularly between 0.5 and 6% by weight in relation to the total weight of the composition, preferably between 0.8 and 5% by weight, and even more advantageously between 1 and 3% by weight.

### Water

The composition according to the invention may optionally comprise water, in a proportion not exceeding 5% by weight, particularly not exceeding 2% by weight, more particularly not exceeding 1% by weight, with respect to the total weight of the composition. Preferably, the composition is anhydrous.

### Adjuvants

In the context of the present invention, the composition may furthermore contain at least one adjuvant selected from those normally used in the cosmetic field, in particular for make-up compositions and/or compositions treating the skin and lips.

By way of examples, mention can be made of preservatives, vitamins, antioxidants, hydrating agents, perfumes, fillers, water-soluble coloring materials or bactericides.

These adjuvants and their concentrations must be such that they do not modify the property required for the composition according to the invention.

The term “water-soluble coloring material” refers, in terms of the invention, to any generally organic, natural or synthetic compound, soluble in an aqueous phase or water-miscible solvents able to color. In particular, the term water-soluble is intended to characterize the ability of a compound to be solubilized in water, measured at 25° C., at a concentration at least equal to 0.1 g/l (obtaining of a macroscopically isotropic and transparent solution, colored or not). This solubility is in particular greater than or equal to 1 g/l.

In terms of water-soluble coloring materials, mention can in particular be made of synthetic or natural water-soluble coloring materials such as for example DC Red 6 (Lithol Rubine Na; CI: 15850), DC Red 22 (CI: 45380), DC Red 28 (CI: 45410, Na salt), DC Red 30 (CI: 73360), DC Red 33 (CI: 17200), DC Red 40 (CI: 16035), FDC Yellow 5 (CI: 19140), FDC Yellow 6 (CI: 15985), DC Yellow 8 (CI: 45350 Na salt), FDC Green 3 (CI: 42053), DC Green 5 (CI: 61570), FDC Blue 1 (CI: 42090).

Given by way of illustration and not limiting of sources of water-soluble coloring material(s) that can be used in the framework of this invention, mention can in particular be made of those of natural origin, such as extracts of carmine, cochineal, beet, grape, carrot, tomato, rocou, paprika, henna, caramel and curcumin.

Thus the water-soluble coloring materials are in particular carminic acid, betanin, anthocyanins, enocyanins, lycopene, bixin, norbixin, capsanthyn, capsorubin, flovoxanthin, lutein, cryptoxanthin, rubixanthin, violaxanthin, riboflavin, roudoxanthin, cantaxanthin, chlorophyll, and mixtures thereof.

It can also be copper sulfate, iron, water-soluble sulfopolyesters, rhodamine, betaine, methylene blue, disodium tartrazine salt and fuchsin disodium salt.

Some of these water-soluble coloring materials are in particular approved from a food standpoint. By way of example of these colorants, more particular mention can be made of the colorants in the carotenoid family, referenced under food codes E120, E162, E163, E160a-g, E150a, E101, E100, E140 and E141.

According to a particularly preferred embodiment, the water-soluble coloring material(s) are selected from the sodium salts of Yellow 6, Yellow 5, Red 6, Red 33 and Red 40.

The product according to the invention can be advantageously used as a make-up product for the skin and/or the lips and/or the cheeks according to the nature of the ingredients used. In particular, the product of the invention can have the form of a foundation, blusher, lipstick, an anti-wrinkle product or eye contour, eye liner, eye shadow, or make-up product for the body.

According to another aspect, the invention also relates to a cosmetic assembly comprising:
i) a receptacle delimiting a compartment, said receptacle being closed by a closing element; and
ii) a composition in accordance with the invention arranged inside said compartment.

The receptacle can be of any suitable shape. It can in particular be the form of a bottle (such as those used for applying liquid lipsticks), a tube, a pot, a case, a box or a casing. Preferably, the receptacle is sealed.

The closing element can be in the form of a removable cap, a lid, a seal, a strip that can be torn, or a capsule, in particular of the type comprising a body secured to the receptacle and a cap hinged on the body. It can also have the form of an element providing the selective closing of the receptacle, in particular a pump or a valve, such as a non-return valve for example. Preferably, the composition according to the invention is disposed in a receptacle of the roll-on type.

Another object of the invention is a method for making up and/or treating the skin and/or lips, in particular the lips and or and/or cheeks, wherein the composition according to the invention is applied to the skin and/or lips and/or cheeks.

The examples hereinafter are given by way of illustration and are not intended to restrict this invention. Unless specified otherwise, the percentages are percentages by weight in relation to the total weight of the composition (% w/w).

Measurement of the stickiness is made by the following protocol:
A sufficient quantity of composition is put in a stainless-steel receptacle 100 microns deep.
The composition is then if necessary levelled before making the measurement.
The receptacles are set to dry at ambient temperature for 1 hour.
The equipment used is a TAXT2i texturometer. The clamp, mounted on the apparatus, grips a 6 mm diameter AU4G cylinder, at the end of which is glued a tip made from smooth beige material imitating the skin, with the same diameter and 2 mm thick.
Between each measurement, the tip is cleaned with ethanol. The measurements are never made at the same point on the sample.
The parameters for the compression tests with the maintenance times are given below:
Approach speed (or pre-speed) 1 mm/s
Speed (from detection of contact) 0.1 mm/s
Force (and corresponding pressure) 0.283 N (i.e. 0.01 MPa)
Contact time 3 seconds
Retraction speed (or post-speed) 0.1 mm/s.

Moreover, the brightness of the compositions is evaluated by observation with the naked eye.

### EXAMPLE 1 OF LIPSTICKS ACCORDING TO THE INVENTION

The following composition according to the invention is prepared according to the following protocol:

The oils, the polyglyceryl-2 triisostearate and the triglycerides are mixed and then heated at 55° C.-60° C.

When the mixture is homogeneous at 55° C.-60° C., the glyceryl trihydroxystearate is introduced under strong stirring;

This is left for 15 minutes under stirring, and then the coloring materials are introduced in the form of a pigmentary homogenate (prior mixing of some of the oils with the pigments); The glycols are added, and mixing is carried out until a homogenous mixture is obtained. The composition is packaged in a receptacle of the gloss type provided with an immersed applicator.

TABLE 1

| Ingredient | Quantity (% w/w) |
|---|---|
| Castor oil | 15.5 |
| (Lipovol CO from Vantage Specialty | |
| Chemicals) | |
| Caprylic/capric triglycerides | 20 |
| (DUB MCT 7030/MB from Stéarineries | |
| Dubois) | |
| Canola oil | QS 100 |
| (Lipex Preact from Aarhuskarlshamn) | |
| Polyglyceryl-2 triisostearate) | 12 |
| (COSMOL 43 V from Nisshin Oillio) | |
| Glyceryl trihydroxystearate | 1.9 |
| (Trihydroxystearin - Thixcin R, from | |
| Elementis Specialties) | |
| Coloring materials (including iron oxides) | 1.2 |
| Pentylene glycol/Caprylyl glycol | 1.5 |

A relatively fluid composition is obtained. Cette composition has a viscosity, measured in accordance with the protocol of the description, of less than 0.5 Pa·s.

This composition is homogenized before use, by simple stirring of the bottle, and does not instantaneously dephase.

This composition can be applied to the skin, the lips and/or the cheeks. After application, it leaves a fine, comfortable, non-sticky, non-glossy to satin and non-fatty deposit.

After storing the composition for at least two weeks at ambient temperature, the composition remains easily rehomogenizable simply by stirring the bottle.

### EXAMPLE 2 OF COMPARATIVE COMPOSITIONS

The following comparative compositions CC1 and CC2 are prepared in accordance with the protocol of example 1, in which the trihydroxystearin of the oily phase of example 1 is replaced by a mixture of glyceryl dibehenate (and) tribehenin (and) glyceryl behenate or by silica, as detailed in the following table.

It should be noted that what is referred to as the “oily phase of example 1” corresponds to all the ingredients except for the glycols.

The appearance of the compositions is evaluated after preparation thereof.

TABLE 2

| | CC1 | CC2 |
|---|---|---|
| Ingredient | Quantity (% w/w) | Quantity (% w/w) |
| Castor oil (Ricinus communis (castor) | 14.8 | 15.5 |
| seed oil) | | |
| (Lipovol CO from Vantage Specialty | | |
| Chemicals) | | |
| Caprylic/capric triglycerides | 19 | 20 |
| (DUB MCT 7030/MB from Stéarineries | | |
| Dubois) | | |
| Canola oil | QS 100 | QS 100 |
| (Lipex Preact from Aarhuskarlshamn) | | |
| Polyglyceryl-2 triisostearate) | 11.5 | 12 |
| (COSMOL 43 V from Nisshin Oillio) | | |
| Glyceryl behenate (and) tribehenin | 7 | — |
| (and) glyceryl behenate | | |
| (Compritol 888 CG ATO from | | |
| Gattefossé) | | |
| Silica | — | 3 |
| (Silica shells de Kobo) | | |
| Coloring materials (including iron | 2 | 2 |
| oxides) | | |
| Appearance of the compositions after | Opaque mixture that | Mixture that dephases |
| preparation | dephases very quickly | very quickly (two- |
| | (two-phase mixture) | phase mixture) |
| | No gelling of the oily | No gelling of the oily |
| | phase | phase |
| Application after rehomogenization | Composition fairly | Composition fairly |
| | difficult to apply (risk of | difficult to apply (risk of |
| | running) and deposit | running) and deposit |
| | obtained very fatty, | obtained very fatty, |
| | very oily | very oily |
| After storage (period of two weeks | Sedimented | Sedimented |
| at ambient temperature) | composition | composition |
| | Rehomogenization | Rehomogenization |
| | possible but requires | possible but requires |
| | very strong stirring of | very strong stirring of |
| | the bottle for several | the bottle for several |
| | minutes: | minutes: |
| | rehomogenization | rehomogenization |
| | very difficult in the | very difficult in the |
| | final packaging | final packaging |

The viscosities of the compositions CC1 and CC2, measured according to the protocol in the description, are less than 1 Pa·s.

1. A cosmetic composition comprising:
glyceryl trihydroxystearate;
at least 50% at least one vegetable oil by weight, relative to the total weight of composition; and
at least one pigmentary coloring material.

2. The composition according to claim 1, which is in a fluid form.

3. The composition according to claim 1, which comprises between 0.5 and 4% by weight glyceryl trihydroxystearate in relation to the total weight of the composition.

4. The composition according to claim 1, wherein the vegetable oil is selected from jojoba oil, castor oil, olive oil, coconut oil, DHA algal oil, ximenia oil, pracaxi oil, coriander seed oil, macadamia oil, passionflower oil, argan oil, sesame oil, sunflower oil, grapeseed oil, avocado oil, Rosa canina oil, apricot kernel oil, flax oil, polymerized flax oil, sweet almond oil, cotton seed oil, soybean oil, rapeseed oil, canola oil, groundnut oil, kaya oil, coconut oil, Marula oil, Camelina oil, wheatgerm oil, corn oil, corn germ oil, rice bran oil, alfalfa oil, poppy seed oil, ambercup squash oil, marrow oil, hazelnut oil, grapeseed oil, blackcurrant oil, evening primrose oil, millet oil, barley oil, quinoa oil, rye oil, safflower oil, candleberry oil, limnanthes oil, black cumin oil, buriti oil, sandal nut oil, Babassu oil, the liquid shea butter fraction, the liquid cocoa butter fraction, and mixtures thereof.

5. The composition according to claim 1, which comprises at least 50% by weight at least one vegetable oil, with respect to the total weight of the composition.

6. The composition according to claim 1, which comprises at least one additional non-volatile polar hydrocarbon oil different from the vegetable oils.

7. The composition according to claim 6, wherein the non-volatile polar hydrocarbon oil different from the vegetable oils is selected from:
monoesters comprising between 17 and 40 carbon atoms in total;
diesters comprising between 18 and 60 carbon atoms in total;
triesters comprising between 25 and 80 carbon atoms in total;
tetraesters, having a total number of carbon of at least 35; and
mixtures thereof.

8. The composition according to claim 1, which comprises at least one non-volatile polar hydrocarbon oil different from the vegetable oils, selected from triesters comprising between 25 and 80 carbon atoms in total.

9. The composition according to claim 6, wherein the concentration of additional non-volatile polar hydrocarbon oil or oils different from the vegetable oils is between 1 and 45% by weight with respect to the total weight of the composition.

10. The composition according to claim 1, characterized in that the concentration of vegetable oil or oils and of additional non-volatile polar hydrocarbon oil or oils represents at least 50% by weight with respect to the total weight of the composition.

11. The composition according to claim 1, which comprises a mixture of vegetable oils.

12. The composition according to claim 1, which comprises less than 5% by weight volatile or non-volatile silicone oil or oils and/or non-polar volatile or non-volatile hydrocarbon oil or oils with respect to the weight of the composition.

13. The composition according to claim 1, which comprises at least one wax different from glyceryl trihydroxystearate, in a proportion not exceeding 1% by weight with respect to the total weight of the composition.

14. The composition according to claim 1, which comprises at least one pasty compound in a proportion not exceeding 5% by weight with respect to the total weight of the composition.

15. The composition according to claim 1, wherein the pigmentary coloring material is selected from mineral pigments, nacres and organic pigments.

16. The composition according to claim 1, which comprises at least one saturated alcohol, linear or branched, comprising 2 to 8 carbon atoms and comprising one or two hydroxyl functions.

17. The composition according to claim 1, which comprises less than 5% water with respect to the total weight of the composition.

18. The composition according to claim 1, which is substantially free from liquid polyester obtained from an unsaturated fatty acid dimer, the fatty acid comprising from 16 to 22 carbon atoms, with a diol.

19. The cosmetic assembly comprising:
i) a receptacle delimiting a compartment, said receptacle being closed by a closing element; and
ii) a composition according to claim 1, disposed inside said compartment.

20. A method for making up and/or treating the skin and/or lips and/or cheeks, wherein the composition according to claim 1 is applied to the skin and/or lips and/or cheeks.
